(19) United States
(12) Patent Application Publication
(10) Pub. No.: US 2011/0092863 A1
(43) Pub. Date: Apr. 21, 2011

(54) LASER TREATMENT DEVICE FOR HAIR GROWTH STIMULATION

(75) Inventors: Jong-Won Kim, Daejeon (KR); Moo-Yeong Lee, Daegu (KR)

(73) Assignee: WON TECHNOLOGY CO., LTD., Daejeon (KR)

(21) Appl. No.: 12/997,786

(22) PCT Filed: Jun. 11, 2009
(86) PCT No.: PCT/KR2009/003137

§ 371 Date: Dec. 13, 2010

(30) Foreign Application Priority Data
Jun. 13, 2008 (KR) 10-2008-0055625

(51) Int. Cl. A61H 1/00; A61N 5/06

(52) U.S. Cl. 601/18

(57) ABSTRACT

Disclosed is a laser treatment device which is designed to stimulate hair growth and prevent hair loss by applying LLLT (Low Level Laser Therapy) to the scalp using laser light of a specified wavelength. The laser treatment device also provides ultra-low frequency mechanical vibrations to the scalp and the combination of light and vibrations is designed to produce synergy. Laser light, which may be blocked by remaining hair, may nonetheless reach the scalp using light-emitting elements attached to the ends of tooth-shaped protrusions. The present laser treatment device is characterised by a bendable applicator having said tooth-shaped protrusions such that it can change shape in accordance with the curvature of the head, which increase the efficiency of light and vibrational energy delivery. The laser treatment device is optimally controlled with a means to measure the light and vibrational energy supplied to the scalp, and the applicator may also be used as a wearable headset, which provide greater simplicity in mounting and removal.

### BACKGROUND OF THE INVENTION

1. Field of the Invention

The present invention relates generally to laser treatment devices for hair growth stimulation and, more particularly, to a laser treatment device for hair growth stimulation which applies low level laser rays and mechanical vibration to the scalp of a patient so as to activate cell tissues of the scalp, thus achieving effects of hair growth stimulation, hair regrowth promotion, hair loss prevention, etc.

2. Description of the Related Art

Hair is an auxiliary organ of the skin. In terms of embryology, hair is a result of a change in the outer layer of the skin. Hence, the hair has a predetermined lifetime and undergoes metabolic change. Hair passes through creation-growth-retrogression-loss and, typically, a period of the lifetime of the hair ranges from 5 to 6 years under normal healthy conditions.

Therefore, to keep thick hair, the hair must have a long lifetime. In addition, when the hair is lost, new hair must rapidly be created and grow.

Recently, it was known that when light having a certain range of wavelength is applied to tissues, the metabolism of the tissues is promoted and the functions of tissues are activated. With this in mind, low level laser therapy (hereinafter, referred to as ‘LLLT’) was proposed and has been developed.

The low level laser therapy generally uses laser rays having wavelengths ranging from 600 μm to 1300 μm and energy intensity ranging from 10 mW/cm² to 1000 mW/cm².

Furthermore, it was known that when such laser rays are applied to tissues, the rays penetrate the tissues and excite molecules of cells, thus promoting the creation of capillary vessels, increasing oxygen storage capacity of blood, promoting creation of collagen and ATP (Adenosine Triphosphate), activating lymph, promoting tissue granulation, and activating phagocytosis.

A hair includes a hair shaft and a hair root. A hair papilla in a base of the hair root is connected to a capillary vessel and a nerve so that nutrition and oxygen are supplied to the hair through the capillary vessel and the nerve to create and grow the hair.

Therefore, when the laser rays are applied to the scalp of a patient, optical energy penetrates the inner skin tissues and subcutaneous tissues, thus activating not only the hair root but also various cell tissues, and promoting metabolism. Thereby, creation of hair and hair growth are promoted, and the lifetime of the hair is extended. As a result, hair loss or a fading white hair phenomenon is prevented, and new hair growth is promoted. As such, the LLLT can heal alopecia of a patient and makes it possible for the patient to have healthy and thick hair.

Typically, a laser diode (hereinafter, referred to as ‘LD’) which emits coherent rays is used as a light source for the LLLT. Furthermore, recently, it was known that a light emitting diode (hereinafter, referred to as an ‘LED’) having the same wavelength has the same effect as that of the LD. Thus, only LEDs may be used, or they may be combined with LDs for LLLT.

Meanwhile, it also was known that when an ultra-low frequency of vibration is applied to tissues, effects of an improvement in blood flow, an increase of the temperature of tissues and activation of the sympathetic nerve can be exhibited by the mechanical stimulation, so that metabolism of the related portion is stimulated and the functions thereof are activated.

Therefore, when this method is applied to the scalp of a patient, hair growth can also be stimulated. Here, it is desirable that the frequency of vibrations range from 1 z to several tens Hz and that the intensity of vibration range from 0.3 W/cm² to 2 W/cm².

The above-mentioned two kinds of therapies are noninvasive, do not require medicine, and are painless therapies. Furthermore, there are no side effects. In particular, when two kinds of therapies are used together, the treatment effect can be further enhanced by their synergistic effect.

Representative examples of a technique which uses both LLLT and vibration therapy for hair growth stimulation were proposed in Korean Utility Model Registration No. 20-335313 and No. 20-0426924.

In Korean U. M. Registration No. 20-335313, a comb-shaped manual applicator was proposed. This applicator includes a single LD for LLLT, a plurality of far infrared LEDs for far infrared therapy, and a single vibration motor. A helmet-shaped fixed type device provided with a plurality of LDs and a plurality of vibration motors was proposed in Korean U. M. Registration No. 20-0426924.

Each of these two conventional techniques provides the LLLT using the LD and the mechanical stimulation effect using the vibration motor to activate scalp tissue. However, in the structure of the applicator disclosed in the conventional techniques, some light rays emitted from the light emitting element are blocked by hair. Thus, the amount of optical energy which reaches the scalp is markedly reduced compared to that of optical energy emitted from the light emitting element. Furthermore, vibrational energy of the motor applied to the scalp is also markedly reduced by the cushion formed by the hair.

Furthermore, because these conventional techniques use many far infrared LDs or LEDs, a large amount of heat is generated, and power consumption is comparatively high.

Meanwhile, the scalp generally has a thickness of several millimeters and covers the skull. Such a scalp is characterized in that it cannot precisely recognize mechanical or thermal stimulation.

Therefore, a patient may not precisely recognize heat generated by the light emitting elements or the intensity of mechanical impact generated by the vibration motor. Particularly, the patient may not correctly recognize, for example, physical damage or a burn attributable to an excessive amount of light irradiated onto the scalp.

Therefore, it is required to sense heat generated on the scalp and the intensity of vibrations applied to the scalp during the operation and control the energy of the light and vibrations applied to the scalp. However, the applicators of the conventional techniques are insufficient in these respects.

As is well known, hair is an auxiliary organ of skin and, in terms of embryology, it is a result of a change in the outer layer of the skin. The creation and growth of hair depends on the health conditions of skin tissues.

Furthermore, it was well known that when laser rays for LLLT are applied to the scalp, optical energy penetrates the interior of the scalp and thus activates the hair roots and the functions of the cell tissues adjacent to the hair roots.

As the wavelength of light increases, the depth to which it penetrates a target is increased. However, the scalp generally has a thickness ranging from 2 mm to 4 mm, and the depth to which the light penetrates the scalp is not required to be greater than 4 mm.

Moreover, if the wavelength of light is excessively long and is within an infrared section, heat generation is increased, thus increasing the probability of cellular tissue being thermally damaged, but the photochemical effect for LLLT is not increased.

Therefore, it is desirable that a laser ray having a wavelength of 1000 nm or less be used in the LLLT for hair growth.

Furthermore, a coherent laser ray is not necessarily required as the laser ray for the LLLT. It was reported that light of an LED having the same wavelength range can also exhibit a satisfactory photochemical effect.

Accordingly, if the number of LEDs used in a unit area is increased and the time for which light is irradiated onto the scalp is increased, the LEDs can provide a level of treatment effect that is similar to that of the LD. Thus, the LD which is comparatively expensive and is not easy to handle is not inevitably required.

Another problem experienced in the LLLT for hair growth is the fact that the scalp is covered with hair other than an area from which a large amount of hair has been already lost.

Therefore, in the conventional techniques, light rays emitted for the LLLT may be blocked by hair and thus optical energy may not reach the surface of the scalp.

This problem is also applied to a term of vibrational energy. In other words, vibrational energy generated from the vibration element is reduced by the absorption effect of hair and thus a sufficient amount of vibrational energy may not be transmitted to the scalp.

Therefore, an applicator which can directly apply light rays or vibrational energy to the scalp without obstruction of hair is required.

Meanwhile, the applicator may have a hand held type or a fixed type as the structure used to apply the LLLT and the vibrational therapy to the scalp together.

In the LLLT or vibrational therapy, the optimal amount of optical or vibrational energy applied to the scalp may vary depending on variables, such as the physical constitution or state of health of the patient, the conditions of scalp tissues and hair, environmental conditions, etc. Therefore, an operator must apply an optimal amount of energy to a corresponding section of the scalp. However, in the case of the hand held type applicator, it is very difficult to achieve this purpose.

Hence, the fixed type applicator which can stay in the correct position and apply a comparatively precise amount of energy to the scalp for a predetermined duration has been mainly used.

However, a cap for supporting such a fixed type applicator is typically large, and the operation of wearing or taking it off inconveniences a user. Thus, a cap having a structure which is comparatively light and convenient to wear or take off is required.

Furthermore, because a required amount of optical and vibrational energies applied to the patient varies depending on variables, such as the physical constitution or state of health of the patient, conditions of scalp tissues and hair, environmental conditions, etc., the optimal values of the amounts of optical and vibrational energies applied to the patient must be clinically determined in advance taking into account these variables.

For this, a structure is required, which is configured such that all variables are previously input in response to corresponding conditions, and the optimal values of the amounts of optical and vibrational energies is calculated, and then the output of the light emitting element and the vibration element are controlled depending on the optimal values.

In addition, a realtime feedback control unit is required, which continuously measures the temperature of a target surface and the conditions of stimulation such that the output power of the applicator is maintained in the optimal state.

Finally, an applied program is required so that even an operator who is not an expert can easily perform all the above-mentioned treatment processes.

### SUMMARY OF THE INVENTION

Accordingly, the present invention has been made keeping in mind the above problems occurring in the prior art, and an object of the present invention is to provide a means for promoting hair growth and preventing hair loss in such a way as to apply optical energy and vibrational energy to the scalp of a patient.

Another object of the present invention is to provide a technique in which the wavelength of a light emitting element is shorter than that of near infrared rays to prevent an excessive heat generation phenomenon, and light emitted from the light emitting element can smoothly reach the scalp without encountering the obstruction of hair.

A further object of the present invention is to provide an automatic control technique such that vibrational energy generated by a vibration element can be directly transmitted to the scalp without the vibrational energy being reduced by the hair and the scalp is prevented from being damaged by excessive vibrations.

Yet another object of the present invention is to provide a laser treatment device for hair growth stimulation in which an applicator unit is mounted to a helmet-shaped or headset-shaped cap so that the user can easily put the device on his/her head or take it off and the cap is convenient to use, and which is comparatively inexpensive.

In order to accomplish the above object, the present invention provides a technique in which light emitting elements, a temperature sensor and a pressure sensor are provided on tips of conical protrusions, and the conical protrusions are arranged on a surface of a flexible applicator, and the applicator is mounted to, for example, a helmet-shaped cap, so that a user can easily put it on his/her head.

Furthermore, the present invention provides a technique in which drive power is supplied to the applicator and the applicator can be controlled such that it is optimally operated.

In addition, the present invention provides a technique in which the wavelength and the output power of the light emitting elements are controlled depending on conditions of the scalp of a patient, thus preventing excessive heat from being generated.

Moreover, the present invention provides a technique which realizes a structure such that the device can be easily put on the head of a patient and output energies of the light emitting elements and a vibration element can be reliably transmitted to the scalp of the patient independent of any outside influence from the surroundings.

In a laser treatment device according to the present invention, when a patient puts the laser treatment device on his/her head, the protrusions provided on the applicator part the hair and come into direct contact with the scalp. Thus, the light emitting elements provided on the tips of the protrusions can directly radiate light onto the scalp.

Furthermore, vibrational energy generated from the vibration element can also be directly applied to the scalp without the vibrational energy being mitigated by the hair.

In addition, a range of the wavelength of the light emitting elements is limited to 1000 nm or less taking into account the thickness of the scalp and penetration characteristics of the light wave, thus preventing heat from being excessively generated and light energy from being wasted.

Moreover, the power consumption required for achieving certain treatment effects is reduced, so that the structure of a power device is simplified, and so that the weight of the power device is reduced.

As well, the applicator unit is easily mounted to the helmet-shaped or headset-shaped cap. Therefore, it is easy to put the treatment device on the head of the patient or remove it from the head, and it is convenient to use. Furthermore, the laser treatment device of the present invention is comparatively inexpensive.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a sectional view of a laser treatment device for hair growth stimulation, according to an embodiment of the present invention;

FIG. 2 is a view showing the laser treatment device put on the head of a user according to the present invention;

FIG. 3 is a view showing an applicator unit of the laser treatment device according to the present invention;

FIG. 4 is a view showing a modification of the applicator unit of the laser treatment device according to the present invention;

FIG. 5 is a view showing the structure of the applicator unit of the laser treatment device according to the present invention;

FIG. 6 is an enlarged sectional view of a protrusion of the applicator unit of the laser treatment device according to the present invention;

FIG. 7 is a view showing a light emitting element and the protrusion of the applicator unit of the laser treatment device according to the present invention;

FIG. 8 is a view illustrating the operation in which light emitted from the light emitting element is radiated onto scalp tissue without being obstructed by hair, according to the present invention;

FIG. 9 is a view illustrating the operation of a vibration element in which vibrational energy is applied to the scalp tissue without being obstructed by hair, according to the present invention;

FIG. 10 is a view showing a power supply circuit for light emitting elements and vibration elements which are arranged on a printed circuit board of the applicator unit according to the present invention; and

FIG. 11 is a block diagram of a power/control unit of the laser treatment device according to the present invention.

### DESCRIPTION OF THE PREFERRED EMBODIMENTS

Hereinafter, a preferred embodiment of the present invention will be described in detail with reference to the attached drawings.

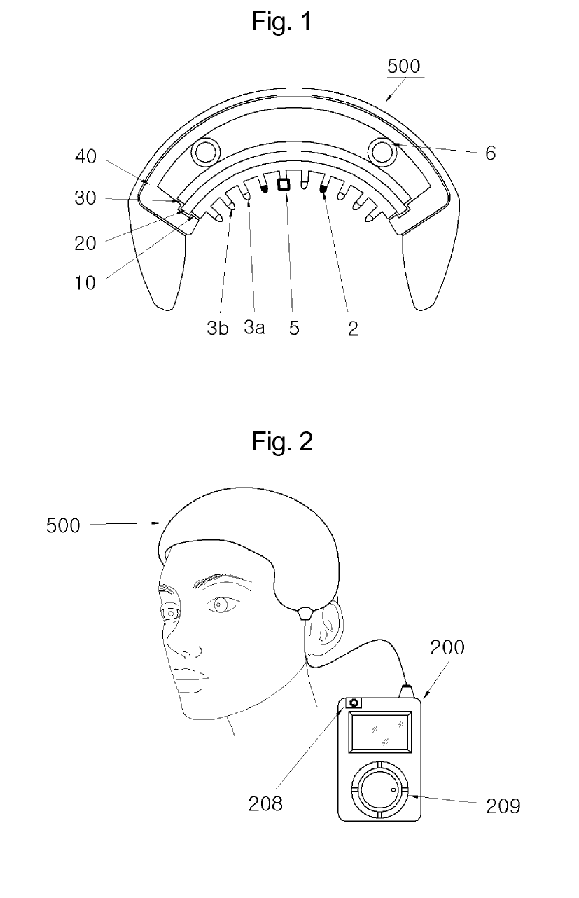

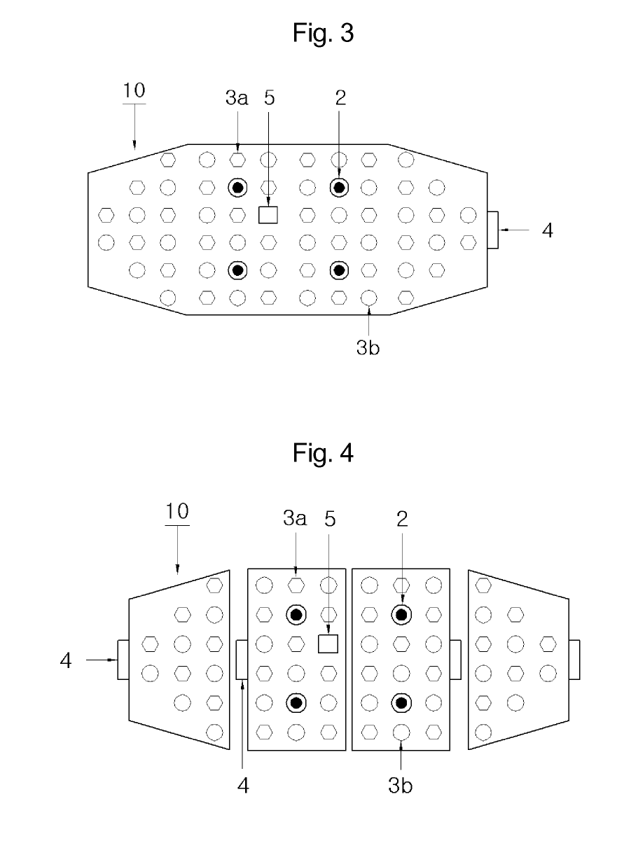

FIG. 1 is a sectional view of a laser treatment device for hair growth stimulation, according to the embodiment of the present invention. FIG. 2 is a view showing the laser treatment device put on the head of a user according to the present invention. FIG. 3 is a view showing an applicator unit of the laser treatment device according to the present invention. FIG. 4 is a view showing a modification of the applicator unit of the laser treatment device according to the present invention.

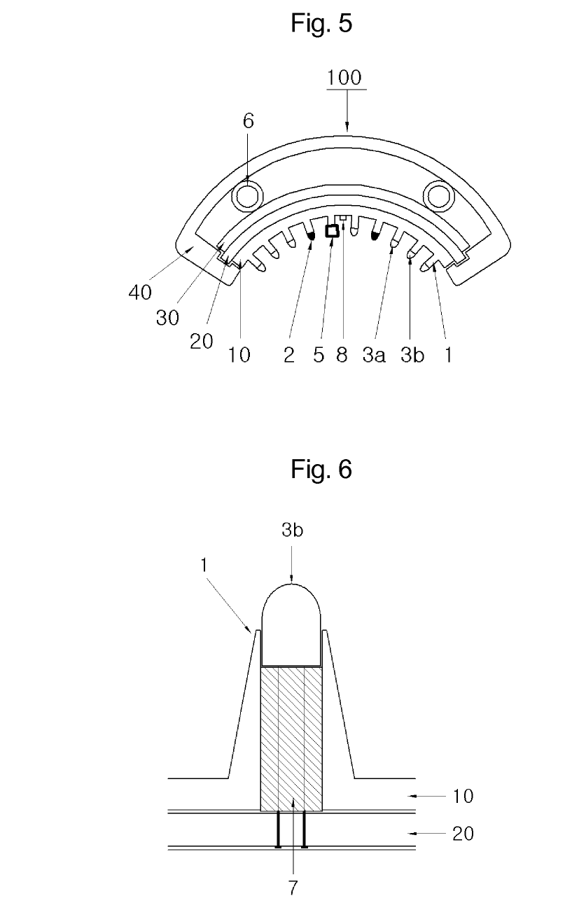

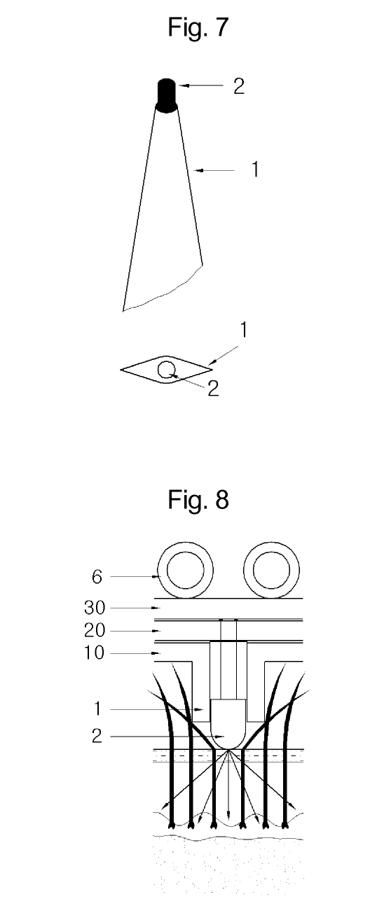

FIG. 5 is a view showing the structure of the applicator unit of the laser treatment device according to the present invention. FIG. 6 is an enlarged sectional view of a protrusion of the applicator unit of the laser treatment device according to the present invention. FIG. 7 is a view showing a light emitting element and the protrusion of the applicator unit of the laser treatment device according to the present invention.

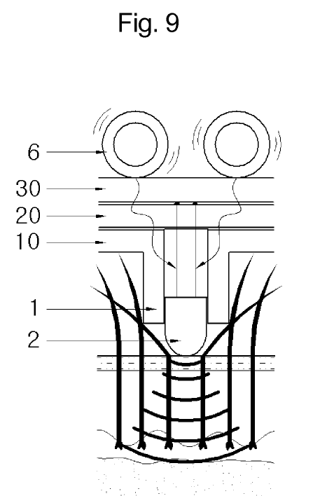

FIG. 8 is a view illustrating the operation in which light emitted from the light emitting element is radiated onto scalp tissue without being obstructed by hair, according to the present invention. FIG. 9 is a view illustrating the operation of a vibration element in which vibrational energy is applied to the scalp tissue without being obstructed by hair, according to the present invention.

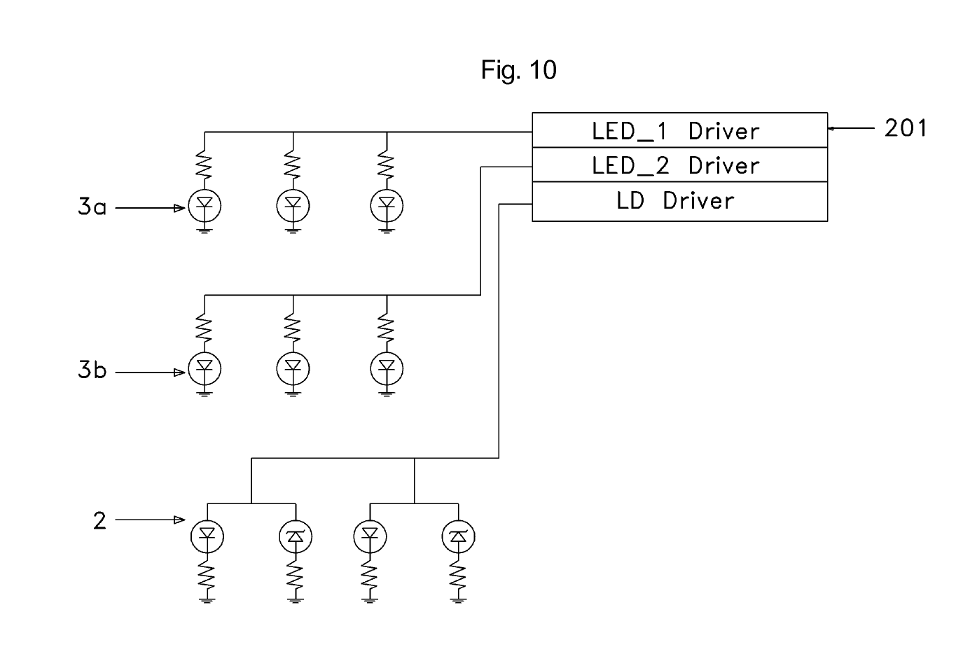

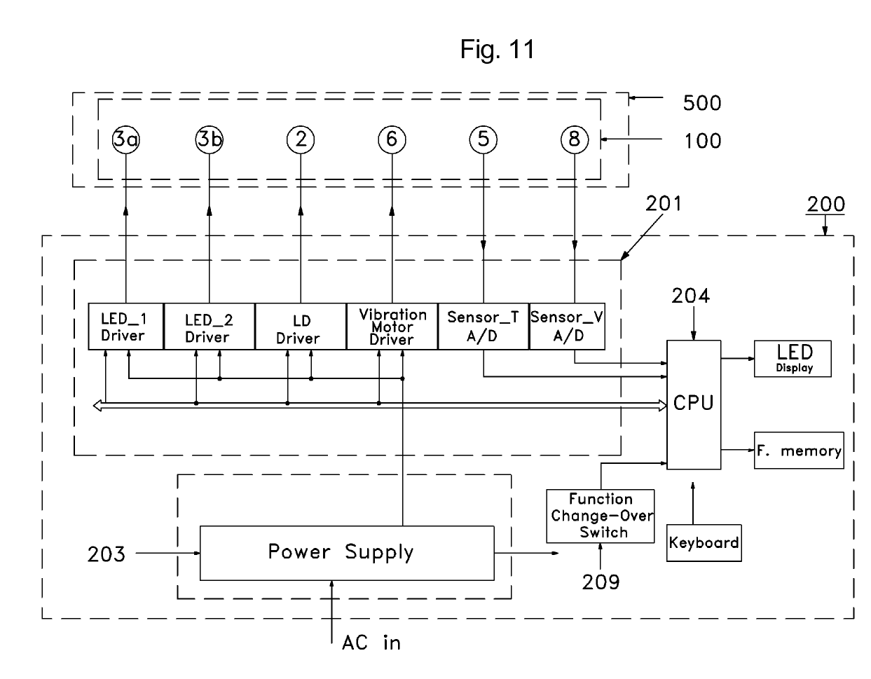

FIG. 10 is a view showing a power supply circuit for light emitting elements and vibration elements which are arranged on a printed circuit board of the applicator unit according to the present invention. FIG. 11 is a block diagram of a power/control unit of the laser treatment device according to the present invention.

As shown in FIG. 1, the laser treatment device for hair growth stimulation according to the present invention includes a cap 500 which has a helmet shape, and an applicator unit 100 which is made of elasto-plastic material and is installed in the cap 500. To use the laser treatment device, the helmet-shaped cap 500 is put on the head of a patient, as shown in FIG. 2. Thereafter, a power/control unit 200 is operated to apply a predetermined amount of optical energy and mechanical energy to the scalp of the patient.

The cap 500 functions to retain the applicator unit 100 on a desired portion of the patient. The cap 500 may adopt various shapes, for example, a helmet shape, a headset shape, a sack shape, etc.

The applicator unit 100 includes a casing 40, an applicator base plate 10, a printed circuit board 20 (hereinafter, referred to as a ‘PCB’), a vibration plate 30 and a plurality of vibration elements 6. The casing 40 is installed in the cap 500 such that an open side of the casing 40 is oriented towards an open side of the cap 500. The applicator base plate 10 has a plurality of protrusions 1 on one surface thereof and is provided on the open side of the casing 40. The applicator base plate 10 is made of flexible plastic. The PCB 20 is installed in the casing 40 behind the applicator base plate 10. The vibration plate 30 is installed in the casing 40 behind the PCB 20. The vibration elements 6 are installed in the casing 40 behind the vibration plate 30 and vibrate the vibration plate 30.

The protrusions 1 are arranged on the surface of the applicator base plate 10. Each protrusion 1 has a pin shape. One of LDs 2 and LEDs 3a and 3b is provided on the end of each protrusion 1. Thus, when the laser treatment device is used, the protrusions 1 come into contact with the scalp of the patient and the LDs 2 and/or LEDs 3a, 3b apply optical energy to the scalp. Furthermore, a temperature sensor 5 is provided on one protrusion 1 which is located at a predetermined position on the applicator base plate 10. When the laser treatment device is used, the temperature sensor 5 comes into contact with the scalp and senses the temperature of the scalp.

When the applicator unit 100 is attached to the head of the patient, as shown in FIGS. 8 and 9, the protrusions 1 part the hair and push the light emitting elements 2, 3a and/or 3b into space between the hairs. Thereby, the light emitting elements 2, 3a and/or 3b provided on the ends of the protrusions 1 come into direct contact with the scalp without hair being interposed between the scalp and the light emitting elements 2, 3a and/or 3b.

The height (length) of each protrusion 1 may vary depending on conditions of a target to be treated. For example, in the case of a completely depilated head (a bald head), several millimeters suffice for the length of each protrusion 1. In the case of thick hair, it is desirable that each protrusion 1 be comparatively long, having a length approximately ranging from 5 mm to 15 mm, so that the protrusion 1 can effectively part hair such that light emitted from the light emitting element 2, 3a, 3b can reach the scalp without being obstructed by hair.

Intervals at which the protrusions 1 are spaced apart from each other may vary depending on light output and light emitting characteristics of the light emitting elements 2, 3a and/or 3b and the lengths of the protrusions 1. Generally, it is effective that the intervals range from 10 mm to 30 mm.

Particularly, in the case of very heavy hair, as shown in FIG. 7, the protrusions 1 each of which has a comb-tooth shape or thin conical shape are used so that the protrusions 1 can easily enter the space between hairs and thus light emitted from the light emitting elements 2, 3a and/or 3b can easily reach the scalp.

Therefore, it is desirable that several kinds of applicator base plates 10 of which protrusions 1 have different shapes and are provided with pin arrays be prepared so that the applicator base plate 10 can be replaced with one having appropriate protrusions 1 depending on conditions of the target to be treated.

As shown in FIG. 8, all the protrusions 1 must be brought into close contact with the scalp. Hence, the applicator base plate 10, the PCB 20 and the vibration plate 30 are made of flexible plastic such that they can vary in the shape in response to the curvature of the head of the patient.

As described above, the flexible PCB 20 is attached to the rear surface of the applicator base plate 10. Lead wires of all the light emitting elements 2, 3a, 3b are inserted into the PCB 20 and are electrically connected to each other by flexible wires.

As shown in the sectional view of FIG. 6, each protrusion 1 preferably has a hollow conical shape. The corresponding light emitting element 2, 3a, 3b or the sensor 5 is inserted into the tip of the protrusion 1, and the lead wire thereof is connected to the PCB 20. Thereafter, the hollow space of the protrusion 1 is filled with silicone resin 7.

Furthermore, it is desirable that the bodies of the protrusions 1 be reliably integrated with the applicator base plate 10, for example, by molding, so that vibrational energy can be directly transmitted from the vibration elements 6 to the tips of the protrusions 1.

As shown in FIG. 3, the applicator base plate 10 may comprise a single plate. Alternatively, the applicator base plate 10 may comprise a plurality of applicator base plates 10 which are elastically connected to each other by connection terminals 4.

In other words, depending on the shape of a target, as shown in FIG. 4, when two through four applicator base plates 10 are separably assembled with each other, it may be more convenient and economical.

An LD 2 or LED 3a, 3b having an output wavelength ranging from 600 mn to 1300 nm is used as each light emitting element 2, 3a, 3b, because it is well known that the LLLT effect is comparatively superior within this wavelength range. Preferably, as shown in FIG. 3, several kinds of light emitting elements which have different wavelengths within a range from 600 mn to 1000 nm are combined.

Here, each light emitting element 2, 3a, 3b outputs power such that the power density thereof on the surface of the scalp ranges from 20 to 1000 mW/cm². Furthermore, an appropriate diverging lens is attached to each light emitting element 2, 3a, 3b so that output light is diffused as wide as possible.

Preferably, as in the embodiment shown in FIG. 3, the light emitting elements 2, 3a and 3b are arrayed such that optical energies of as many wavelengths as possible are uniformly applied to a target portion. An interval between the light emitting elements 2, 3 and 3b is determined depending on the output of the corresponding light emitting elements 2, 3a and/or 3b and the number of combined elements and, preferably, the interval ranges from 10 mm to 30 mm.

FIG. 3 illustrates the first embodiment in which the LEDs 3a having a wavelength of 660 nm, the LEDs 3b having a wavelength of 880 nm, and the LD2 having a wavelength of 904 nm are arranged on the applicator base plate 10 having an area of 344 cm².

One or more vibration elements 6 are provided at predetermined positions on the rear surface of the applicator base plate 10. As shown in FIG. 9, the output power of the vibration elements 6 is transmitted from the applicator base plate 10 to the scalp of the patient via the protrusions 1.

Mechanical or piezoelectric devices which can vibrate the vibration plate 30 and the applicator base plate 10 with the frequency of vibrations ranging from 1 Hz to several tens Hz and are used as the vibration elements 6. Preferably, a mechanical vibrator which is operated by a low-speed eccentric motor is used as the vibration element 6 so that vibrational energy ranging from 0.3 to 2 W/cm² can be applied to the scalp of the patient.

Furthermore, it is preferable that the vibration elements 6 are located on the opposite ends of the applicator base plate 10, as shown I FIG. 5, so that vibrational energy can be uniformly transmitted to the overall area of the scalp.

As shown in FIG. 10, the light emitting elements 2, 3a, 3b and the vibration elements 6 which are provided on the applicator base plate 10 are connected to each other in series or in parallel and electrically connected to the power/control unit 200.

The light emitting elements 2, 3a and 3b receive drive power from the PCB 20 connected to the terminal 4. The vibration elements 6 receive their drive power through connection wires extending from the terminal 4.

The power/control unit 200 includes a power supply 203 which supplies power to all the elements 2, 3a, 3b and 6, and a control unit 201 which controls the elements 2, 3a, 3b and 6. An on/off power switch 208, a function change-over switch 209 and an LCD indicator are provided on an outer casing of the power/control unit 200.

The function change-over switch 209 changes over a mode by which drive power is applied to the elements 2, 3a, 3b and 6, between two modes including an automatic mode and a manual mode.

Here, in manual mode, the operational array, a period of on/off operation and the intensity of vibrations are selected manually. In automatic mode, with regard to the light emitting elements 2, 3a and 3b and the vibration elements 6, selection of the operational array, selection of a period of on/off operation, selection of the intensity of vibration and a reservation or cancel function are automatically conducted according to a preset routine.

Meanwhile, the temperature sensor 5 is provided on the central portion of the applicator base plate 10 to sense the temperature of the scalp of the patient during the operation.

The temperature sensor 5 is configured by attaching a temperature sensing device, in place of the light emitting element 2, 3a or 3b, to the tip of the corresponding protrusion 1. When the laser treatment device is used, the temperature sensor 5 comes into close contact with the surface of the scalp of the patient, senses the temperature of the contact portion of the scalp, creates an electric signal for the sensed temperature, and then transmits the electric signal to the control unit 201 of the power/control unit 200 through the PCB 20.

The control unit 201 digitizes the electric signal and transmits it to a CPU 204 which is a microprocessor. The CPU 204 processes the digitized electric signal according to a routine which has been previously stored. Thereafter, a temperature value is displayed on the LCD indicator. When the temperature value reaches the uppermost limit, a buzzer or an indication lamp is operated and the drive circuits of the light emitting elements 2, 3a and 3b are automatically interrupted.

Furthermore, a vibration sensor 8 is attached at a predetermined position to the applicator base plate 10 to measure mechanical impact or the intensity of vibrations which are applied to the scalp during the operation. The vibration sensor 8 which comprises a piezoelectric device creates an electric signal for the sensed vibration and then transmits the electric signal to the control unit 201 of the power/control unit 200 via the PCB 20.

The control unit 201 digitalizes this electric signal and transmits it to the CPU 204. The CPU 204 processes the digitalized electric signal according to a routine which has been previously stored. When the intensity of the vibrations reaches the upper limit, the buzzer or the indication lamp is operated and the drive circuit of the vibration element 6 is automatically interrupted.

As described above, in a laser treatment device according to the present invention, all light emitting elements are located on the tips of protrusions of an applicator unit so that the light emitting elements can come into direct contact with the scalp of a patient, thus preventing the loss of optical energy. In addition, a transmission rate of vibrational energy from a vibration motor to the scalp is prevented from being reduced.

Furthermore, depending on characteristics of a target portion of the scalp, several applicator base plates may be combined and can be easily removably mounted to a cap having a headset shape or a helmet shape. In other words, the laser treatment device of the present invention has superior adaptability.

Moreover, the power consumption required to achieve a certain treatment effect is reduced, so that power rates are reduced, the structure of a power device is simplified, and the weight of the power device is reduced.

As a result, the present invention can provide the laser treatment device which exhibits the enhanced treatment effect, is simple to use, and is comparatively inexpensive.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A laser treatment device for hair growth stimulation, comprising:
a cap having a U shape, the cap being put on a head of a patient; and
an applicator unit installed in the cap, the applicator unit comprising a plurality of protrusions each of which has a light emitting element on a tip thereof, so that the cap is put on the head of the patient, the protrusions push through hair and the light emitting elements of the protrusions emit light onto a scalp of the head, the protrusions applying vibrations to the scalp.

2. The laser treatment device as set forth in claim 1, wherein the applicator unit comprises:
a casing installed in the cap such that an open side of the casing is oriented towards an open side of the cap;
an applicator base plate provided on the open side of the casing, with the protrusions provided on a corresponding surface of the applicator base plate;
a printed circuit board installed in the casing behind the applicator base plate, with the light emitting elements electrically mounted to the printed circuit board; and
a vibration unit installed in the casing behind the printed circuit board, the vibration unit vibrating the protrusions.

3. The laser treatment device as set forth in claim 2, wherein the vibration unit comprises:
a vibration plate installed in the casing behind the printed circuit board; and
a vibration element installed in the casing behind the vibration plate, the vibration element vibrating the vibration plate so that the protrusions vibrate.

4. The laser treatment device as set forth in claim 3, wherein the vibration element applies vibrational energy ranging from 0.3 W/cm² to 2 W/cm² to the scalp.

5. The laser treatment device as set forth in claim 2, wherein the applicator base plate is flexible and variable in shape so that the tips of the protrusions come into contact with the scalp during operation.

6. The laser treatment device as set forth in claim 5, wherein the printed circuit board and the vibration plate are flexible and variable in shape in response to the applicator base plate.

7. The laser treatment device as set forth in claim 2, wherein the applicator base plate comprises a plurality of applicator base plates which are electrically assembled with each other by a connection terminal provided between the applicator base plates.

8. The laser treatment device as set forth in claim 1, wherein each of the protrusions is reduced in thickness from a root end to a tip thereof.

9. The laser treatment device as set forth in claim 8, wherein each of the protrusions has either of a comb-tooth shape or a conical shape.

10. The laser treatment device as set forth in claim 8, wherein each of the protrusions has a length from 5 mm to 15 mm such that when the protrusions come into contact with the scalp to provide treatment, the protrusions part the hair to prevent light emitted from the light emitting elements from being blocked by the hair.

11. The laser treatment device as set forth in claim 1, wherein the protrusions are spaced apart from each other by a distance ranging from 10 mm to 30 mm.

12. The laser treatment device as set forth in claim 1, wherein each of the light emitting elements comprises at least one of a laser diode (LD) and a light emitting diode (LED).

13. The laser treatment device as set forth in claim 12, wherein each of the LD and the LED has an output wavelength ranging from 600 nm to 1300 nm.

14. The laser treatment device as set forth in claim 12, wherein each of the LD and the LED outputs power such that a power density thereof on a surface of the scalp ranges from 20 to 1000 mW/cm².

15. The laser treatment device as set forth in claim 1, further comprising:
a power/control unit electrically connected to the applicator unit to supply power to the applicator unit and control operation of the applicator unit.

16. The laser treatment device as set forth in claim 15, further comprising:
a temperature sensor provided on a tip of one selected from the protrusions, the temperature sensor being electrically connected to the power/control unit through the PCB to sense a temperature of the surface of the scalp and transmit a signal for the sensed temperature to the power/control unit.

17. The laser treatment device as set forth in claim 16, wherein the power/control unit displays a temperature value to an outside using the temperature signal transmitted from the temperature sensor and, when the temperature value reaches an upper limit, creates a warning signal and interrupts the operation of the light emitting elements.

18. The laser treatment device as set forth in claim 15, further comprising:
a vibration sensor provided on the applicator base plate, the vibration sensor being electrically connected to the power/control unit by the PCB to measure mechanical impact or intensity of vibrations applied to the scalp and transmit a signal representing the measured impact or intensity to the power/control unit.

19. The laser treatment device as set forth in claim 18, wherein the power/control unit receives the signal transmitted from the vibration sensor and, when the intensity of vibrations reaches an upper limit, creates a warning signal and interrupts the operation of the vibration element.

20. The laser treatment device as set forth in claim 1, wherein the cap has one of a headset shape or a helmet shape.
